(19) United States
(12) Patent Application Publication
(10) Pub. No.: US 2022/0383217 A1
(43) Pub. Date: Dec. 1, 2022

(54) RISK EVALUATING SYSTEM, RISK EVALUATING METHOD, AND COMPUTER PROGRAM

(71) Applicant: National University Corporation Tokai National Higher Education and Research System, Nagoya-shi (JP)

(72) Inventors: Masakazu UEMURA, Nagoya-shi, Aichi (JP); Yoshimasa NAGAO, Nagoya-shi, Aichi (JP); Kouichi TANABE, Nagoya-shi (JP)

(21) Appl. No.: 17/773,704

(22) PCT Filed: Oct. 26, 2020
(86) PCT No.: PCT/JP2020/040057

§ 371 Date: May 2, 2022

(30) Foreign Application Priority Data
Oct. 30, 2019 (JP) 2019-197314

(51) Int. Cl. G06Q 10/06; G16H 15/00

(52) U.S. Cl. CPC G06Q 10/0635; G16H 15/00

(57) ABSTRACT

A negligence term score storage stores, for each of a plurality of words, a negligence term score that is an index representing the likelihood of appearance in a report indicating that an event has occurred due to negligence of an organization. A severity term score storage stores, for each of a plurality of words, a seriousness term score that is an index representing the likelihood of appearance in a report indicating that a serious event has occurred. A score deriver extracts the plurality of words described in the report to be analyzed, and derives a risk score that is an index representing the magnitude of the risk of the organization on the basis of the negligence term score of each of the extracted words, the seriousness term score of each of the extracted words, and a weight in risk evaluation determined in advance.

### TECHNICAL FIELD

The present disclosure relates to data processing technology, and particularly to a risk evaluation system, a risk evaluation method, and a computer program.

### BACKGROUND ART

In a medical institution, a report called an incident report is created. The incident refers to a medical accident, a medical error, or an event that may lead to them at a daily medical site. The incident report is utilized to analyze the incident to prevent recurrence of similar incidents and to prevent occurrence of medical accidents and medical errors in advance. The present applicant has proposed technology for accurately classifying incident reports in the following Patent Literature 1.

### CITATION LIST

### Patent Literature

[Patent Literature 1] JP 2018-081334 A

### SUMMARY OF INVENTION

### Technical Problem

Until now, the magnitudes of risks held by a plurality of medical institutions have not been sufficiently compared between the medical institutions. Further, the magnitudes of risks held by a plurality of departments in a medical institution have not been sufficiently compared between the departments. The present inventor has considered that the magnitude of a risk held by an organization can be accurately estimated by analyzing the incident report.

The present disclosure has been made on the basis of the above idea of the present inventor, and one object is to provide technology for accurately evaluating the magnitude of a risk of an organization, on the basis of a report for reporting an event having occurred in the organization.

### Solution to Problem

In order to solve the above problem, a risk evaluation system according to an aspect of the present disclosure is a system for analyzing a report for reporting an event having occurred in an organization and evaluating the magnitude of a risk of the organization. The risk evaluation system includes: a first storage structured to store, for each of a plurality of words, a negligence term score that is an index representing the likelihood of appearance in a report indicating that an event has occurred due to negligence of the organization; a second storage structured to store, for each of a plurality of words, a seriousness term score that is an index representing the likelihood of appearance in a report indicating that a serious event has occurred; and a score deriver structured to extract the plurality of words described in the report to be analyzed and derive a risk score that is an index indicating the magnitude of the risk of the organization, on the basis of the negligence term score of each of the extracted words, the seriousness term score of each of the extracted words, and a weight in risk evaluation determined in advance for each of the negligence of the organization and the seriousness of the event.

Another aspect of the present disclosure is a risk evaluation method. The method is a method in which a computer accessible to a first storage and a second storage analyzes a report for reporting an event having occurred in an organization and evaluates the magnitude of a risk of the organization. The method includes: causing the first storage to store, for each of a plurality of words, a negligence term score that is an index representing the likelihood of appearance in a report indicating that an event has occurred due to negligence of the organization, causing the second storage to store, for each of a plurality of words, a seriousness term score that is an index representing the likelihood of appearance in a report indicating that a serious event has occurred, extracting the plurality of words described in the report to be analyzed; and deriving a risk score that is an index representing the magnitude of the risk of the organization, on the basis of the negligence term score of each of the extracted words, the seriousness term score of each of the extracted words, and a weight in risk evaluation determined in advance for each of the negligence of the organization and the seriousness of the event.

Note that arbitrary combinations of the above components and modifications of the expressions of the present disclosure in devices, computer programs, recording media storing the computer programs, and the like are also effective as aspects of the present disclosure.

### Advantageous Effects of Invention

According to the present disclosure, it is possible to accurately evaluate the magnitude of a risk of an organization, on the basis of a report for reporting an event having occurred in the organization.

### BRIEF DESCRIPTION OF DRAWINGS

FIG. 1 is a diagram schematically illustrating a method for determining a negligence term score.

FIG. 2 is a diagram schematically illustrating a method for determining a severity term score.

FIG. 3 is a diagram schematically illustrating a method for determining an impact term score.

FIG. 4 is a block diagram illustrating functional blocks of a risk evaluation device according to a first embodiment.

FIG. 5 is a flowchart illustrating a flow of negligence score calculation processing.

FIG. 6 is a diagram illustrating weights of risk evaluation factors in a hospital.

FIG. 7 is a diagram illustrating candidates for a method for calculating a risk score.

FIG. 8 is a diagram illustrating a correlation between rank evaluation by GRM and rank evaluation by each calculation method.

FIG. 9 is a diagram illustrating an example of a risk evaluation result according to the first embodiment.

FIG. 10 is a diagram illustrating an example of a risk evaluation result according to the first embodiment.

FIG. 11 is a scatter diagram illustrating a relation between a report amount and a risk score.

FIG. 12 is a diagram illustrating a specific example of risk deviation derivation.

FIG. 13 is a diagram illustrating an example of a risk evaluation result according to a second embodiment.

FIG. 14 is a diagram illustrating an example of a risk evaluation result according to the second embodiment.

FIG. 15 is a diagram illustrating an example of a risk evaluation result according to the second embodiment.

FIG. 16 is a diagram illustrating an example of a risk evaluation result according to the second embodiment.

FIG. 17 is a diagram illustrating an example of a risk evaluation result according to the second embodiment.

FIG. 18 is a diagram illustrating an example of a risk evaluation result according to the second embodiment.

### DESCRIPTION OF EMBODIMENTS

### First Embodiment

In a first embodiment, technology for accurately evaluating the magnitude of a risk held by a medical institution on the basis of an incident report created by the medical institution is proposed. With this technology, for example, the magnitude of risks held by organizations of various granularities such as medical institutions and departments in medical institutions can be visualized, and risk comparison between organizations can be performed. Hereinafter, the medical institution will be described as a hospital.

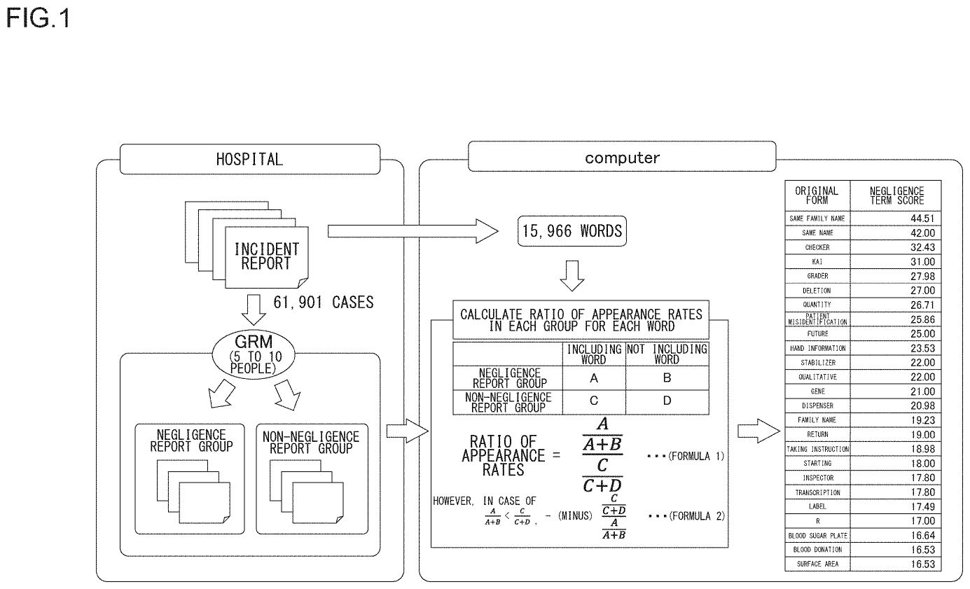

In the first embodiment, a negligence term score, a severity term score, and an impact term score are used as indices corresponding to a “feature degree” described in Patent Literature 1. FIG. 1 schematically illustrates a method for determining the negligence term score. The negligence term score is an index representing the likelihood of appearance in an incident report indicating that an incident has occurred due to the negligence of the hospital (in other words, a medical worker), for each of a plurality of words.

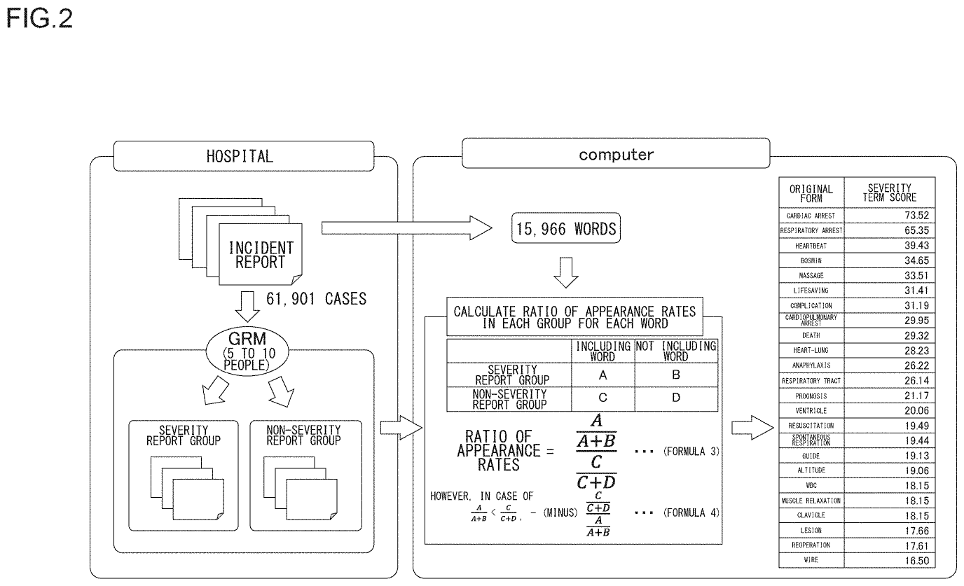

FIG. 2 schematically illustrates a method for determining the severity term score. The severity term score is an index representing the likelihood of appearance in an incident report indicating that a severe situation, that is, a serious event has occurred in a patient, specifically, an incident report indicating that the patient has become severe (that a serious situation has occurred in the patient due to the incident), for each of a plurality of words.

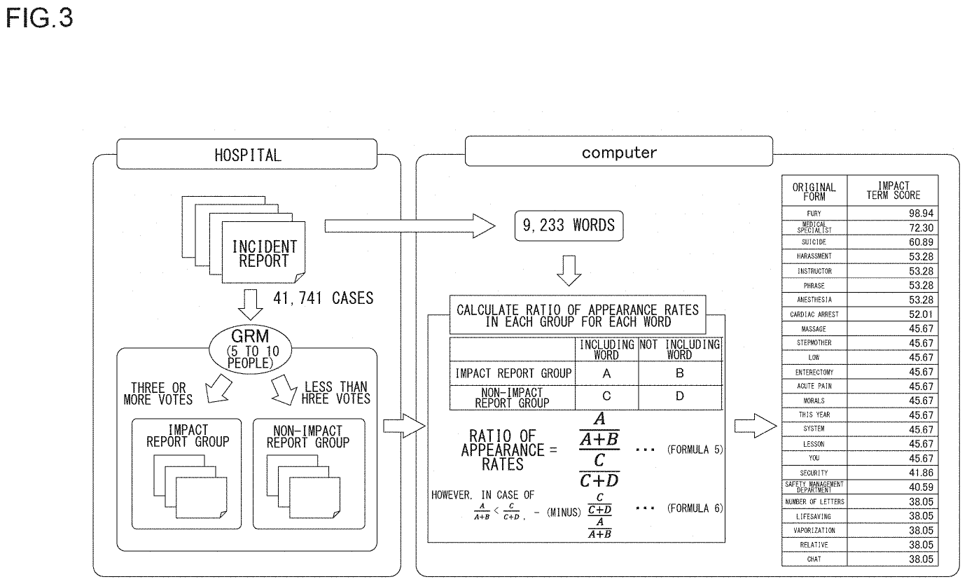

FIG. 3 schematically illustrates a method for determining the impact term score. The impact term score is an index representing the likelihood of appearance in an incident report in which a person has determined that important contents are described, in other words, an incident report in which a person has determined that careful consideration or some action is necessary, for each of a plurality of words.

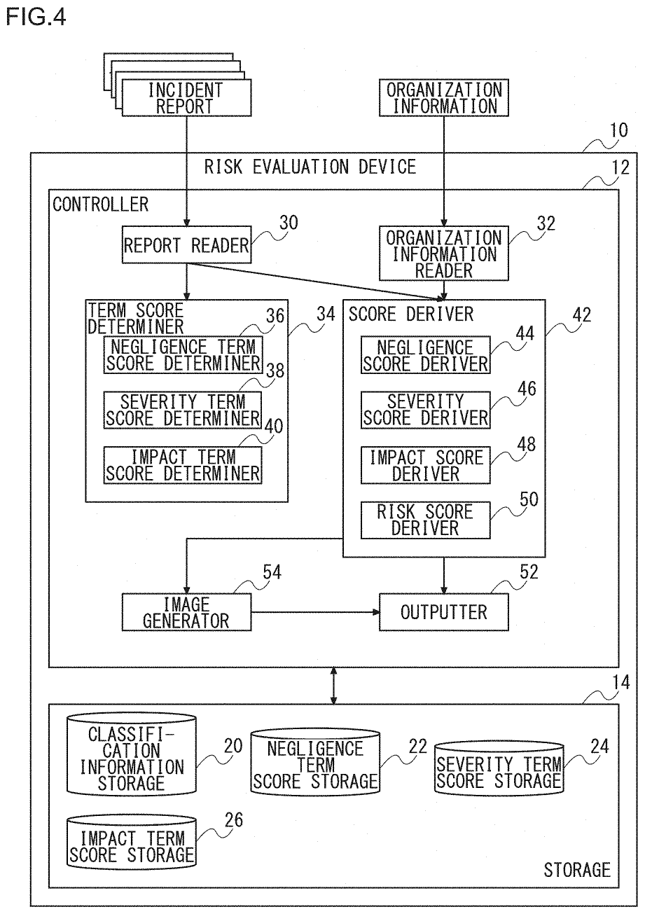

FIG. 4 is a block diagram illustrating functional blocks of a risk evaluation device according to the first embodiment. Each block illustrated in the block diagram of the present specification can be realized by elements including a processor, a CPU, and a memory of a computer, an electronic circuit, and a mechanical device in terms of hardware, and can be realized by a computer program or the like in terms of software. However, here, functional blocks realized by cooperation of these are illustrated. Therefore, those skilled in the art will understand that these functional blocks can be realized in various forms by combining hardware and software.

The risk evaluation device 10 is an information processing device that analyzes the incident report and evaluates the magnitude of the risk held by the hospital or each department in the hospital. The risk evaluation device 10 includes a controller 12 and a storage 14. The controller 12 executes various data processing for risk evaluation. The storage 14 stores data to be referred to or updated by the controller 12. The storage 14 includes a classification information storage 20, a negligence term score storage 22, a severity term score storage 24, and an impact term score storage 26.

The classification information storage 20 stores classification information indicating whether or not each of a plurality of incident reports created in the hospital corresponds to an incident report indicating that an incident has occurred due to negligence of the hospital (in other words, a medical worker). A set of corresponding incident reports is referred to as a “negligence report group”, and a set of non-corresponding incident reports is referred to as a “non-negligence report group”. As illustrated in FIG. 1, a GRM (General Risk Manager or Medical Safety Manager) in the hospital checks contents of the plurality of incident reports and determines whether each incident report corresponds to the negligence report group or the non-negligence report group. Classification information indicating this determination result is stored in the classification information storage 20. For example, the classification information storage 20 may store identification information (a report number or the like) of one or more incident reports corresponding to the negligence report group and identification information of one or more incident reports corresponding to the non-negligence report group.

In addition, the classification information storage 20 stores classification information indicating whether or not each of the plurality of incident reports created in the hospital corresponds to an incident report indicating that the patient has become severe. A set of corresponding incident reports is referred to as a “severity report group”, and a set of non-corresponding incident reports is referred to as a “non-severity report group”. As illustrated in FIG. 2, the GRM of the hospital checks the contents of the plurality of incident reports and determines whether each incident report corresponds to the severity report group or the non-severity report group. Classification information indicating this determination result is stored in the classification information storage 20. For example, the classification information storage 20 may store identification information of one or more incident reports corresponding to the severity report group and identification information of one or more incident reports corresponding to the non-severity report group.

In addition, the classification information storage 20 stores classification information indicating whether or not each of the plurality of incident reports created in the hospital corresponds to an incident report in which a person has determined that important contents are described. A set of corresponding incident reports is referred to as an “impact report group”, and a set of non-corresponding incident reports is referred to as a “non-impact report group”. As illustrated in FIG. 3, the GRM of the hospital checks the contents of the plurality of incident reports and determines whether each incident report corresponds to the impact report group or the non-impact report group. Classification information indicating this determination result is stored in the classification information storage 20. For example, the classification information storage 20 may store identification information of one or more incident reports corresponding to the impact report group and identification information of one or more incident reports corresponding to the non-impact report group.

Note that, in the embodiment, eight GRMs are caused to vote whether or not each incident report corresponds to the impact report group. In addition, incident reports with three or more votes that correspond to the impact report group are classified into the impact report group, and incident reports with less than three votes are classified into the non-impact report group.

Returning to FIG. 4, the negligence term score storage 22 stores a negligence term score of each of a plurality of words extracted from the incident report. The severity term score storage 24 stores a severity term score of each of the plurality of words extracted from the incident report. The impact term score storage 26 stores an impact term score of each of the plurality of words extracted from the incident report.

The controller 12 includes a report reader 30, an organization information reader 32, a term score determiner 34, a score deriver 42, an outputter 52, and an image generator 54. A computer program including a plurality of modules corresponding to the plurality of functional blocks may be stored in a recording medium and installed in the storage of the risk evaluation device 10 via the recording medium. Alternatively, the computer program may be installed in the storage of the risk evaluation device 10 via a network. The CPU of the risk evaluation device 10 may perform the functions of the plurality of functional blocks by reading and executing the computer program in a main memory.

The report reader 30 reads data (for example, text data) of the incident report from an electronic file in which the incident report is recorded. Data items of the incident report include a report number, creator information (including job categories such as doctors and nurses), an occurrence date (date when the incident occurs), a department name (department name where the incident occurs), and free description. The risk evaluation device 10 scores the magnitude of the risk on the basis of the description in the free description of the incident report.

The organization information reader 32 reads organization information from an electronic file in which information regarding each department of the hospital (also referred to as “organization information”) is recorded. The organization information includes a name (department name) and the number of people belonging to each of a plurality of departments in the hospital. The organization information may include names and the number of people belonging to a plurality of departments provided in each hospital for each of a plurality of hospitals.

The term score determiner 34 includes a negligence term score determiner 36 that determines the negligence term score of each of the plurality of words, a severity term score determiner 38 that determines the severity term score of each of the plurality of words, and an impact term score determiner 40 that determines the impact term score of each of the plurality of words.

A method for determining the negligence term score will be described with reference to FIG. 1. The negligence term score determiner 36 extracts a plurality of words (15966 words in the example of FIG. 1) included in the plurality of incident reports read by the report reader 30. For example, the negligence term score determiner 36 may extract nouns (or nouns and verbs) from sentences described in free description fields of a plurality of incident reports using known technology such as morphological analysis. In addition, the negligence term score determiner 36 refers to the classification information stored in the classification information storage 20 to identify whether each of the plurality of incident reports read by the report reader 30 corresponds to the negligence report group or the non-negligence report group.

Here, a word to be a target for obtaining the negligence term score is referred to as a target word. The negligence term score determiner 36 specifies the number of reports A including the target word in the free description field in the negligence report group, the number of reports B not including the target word in the free description field in the negligence report group, the number of reports C including the target word in the free description field in the non-negligence report group, and the number of reports D not including the target word in the free description field in the non-negligence report group. The negligence term score determiner 36 calculates a ratio of appearance rates of the target word in the negligence report group and the non-negligence report group as the negligence term score of the target word, according to Formula 1 or Formula 2 in FIG. 1.

The negligence term score determiner 36 executes the above processing with each of the plurality of extracted words sequentially as a target word, and calculates the negligence term score of each word. The negligence term score determiner 36 stores the plurality of words and the negligence term score of each word in association with each other in the negligence term score storage 22.

As represented by Formula 1 and Formula 2, the negligence term score is a numerical value of less than −1 or a numerical value of 1 or more. When the negligence term score of a word is larger, it means that the word is more likely to appear in the negligence report group than the non-negligence report group. In the example of FIG. 1, a word “same family name” indicates that the appearance rate in the negligence report group is about 45 times the appearance rate in the non-negligence report group. In addition, when the negligence term score of a word is smaller, it means that the word is more likely to appear in the non-negligence report group than the negligence report group.

A method for determining the severity term score will be described with reference to FIG. 2. Similarly to the negligence term score determiner 36, the severity term score determiner 38 extracts a plurality of words (15966 words in the example of FIG. 2) included in the plurality of incident reports read by the report reader 30. In addition, the severity term score determiner 38 refers to the classification information stored in the classification information storage 20 to identify whether each of the plurality of incident reports read by the report reader 30 corresponds to the severity report group or the non-severity report group.

Here, a word to be a target for obtaining the severity term score is referred to as a target word. The severity term score determiner 38 specifies the number of reports A including the target word in the free description field in the severity report group, the number of reports B not including the target word in the free description field in the severity report group, the number of reports C including the target word in the free description field in the non-severity report group, and the number of reports D not including the target word in the free description field in the non-severity report group. The severity term score determiner 38 calculates a ratio of appearance rates of the target word in the severity report group and the non-severity report group as the severity term score of the target word, according to Formula 3 or Formula 4 of FIG. 2.

The severity term score determiner 38 executes the above processing with each of the plurality of extracted words sequentially as a target word, and calculates the severity term score of each word. The severity term score determiner 38 stores the plurality of words and the severity term score of each word in association with each other in the severity term score storage 24.

Similarly to the negligence term score, the severity term score is a numerical value of less than −1 or a numerical value of 1 or more. When the severity term score of a word is larger, it means that the word is more likely to appear in the severity report group than the non-severity report group. In the example of FIG. 2, a word “cardiac arrest” indicates that the appearance rate in the severity report group is about 74 times the appearance rate in the non-severity report group. In addition, when the severity term score of a word is smaller, it means that the word is more likely to appear in the non-severity report group than the severity report group.

A method for determining the impact term score will be described with reference to FIG. 3. Similarly to the negligence term score determiner 36, the impact term score determiner 40 extracts a plurality of words (14000 words in the example of FIG. 3) included in the plurality of incident reports read by the report reader 30. In addition, the impact term score determiner 40 refers to the classification information stored in the classification information storage 20 to identify whether each of the plurality of incident reports read by the report reader 30 corresponds to the impact report group or the non-impact report group.

Here, a word to be a target for obtaining the impact term score is referred to as a target word. The impact term score determiner 40 specifies the number of reports A including the target word in the free description field in the impact report group, the number of reports B not including the target word in the free description field in the impact report group, the number of reports C including the target word in the free description field in the non-impact report group, and the number of reports D not including the target word in the free description field in the non-impact report group. The impact term score determiner 40 calculates a ratio of appearance rates of the target word in the impact report group and the non-impact report group as the impact term score of the target word, according to Formula 5 or Formula 6 in FIG. 3.

The impact term score determiner 40 executes the above processing with each of the plurality of extracted words sequentially as a target word, and calculates the impact term score of each word. The impact term score determiner 40 stores the plurality of words and the impact term score of each word in association with each other in the impact term score storage 26.

Similarly to the negligence term score, the impact term score is a numerical value of less than −1 or a numerical value of 1 or more. When the impact term score of a word is larger, it means that the word is more likely to appear in the impact report group than the non-impact report group. In the example of FIG. 3, a word “fury” indicates that the appearance rate in the impact report group is about 99 times the appearance rate in the non-impact report group. In addition, when the impact term score of a word is smaller, it means that the word is more likely to appear in the non-impact report group than the impact report group.

The score deriver 42 derives various scores quantitatively indicating the magnitude of the risk held by the hospital and each department in the hospital. The score deriver 42 includes a negligence score deriver 44, a severity score deriver 46, an impact score deriver 48, and a risk score deriver 50.

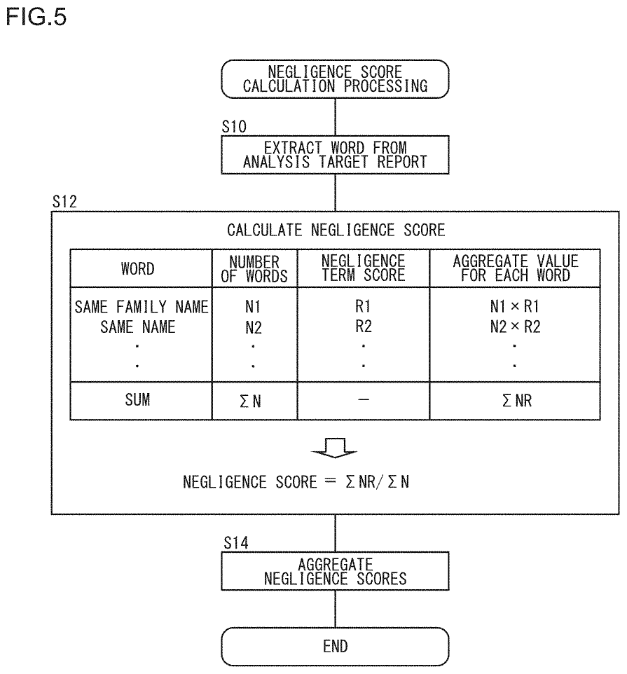

The negligence score deriver 44 derives the negligence score, which is an index representing the magnitude of the risk from the viewpoint of the negligence of the hospital (in other words, the negligence of the medical worker), on the basis of the negligence term score stored in the negligence term score storage 22. FIG. 5 is a flowchart illustrating a flow of negligence score calculation processing. The negligence score deriver 44 extracts a plurality of words described in an analysis target incident report (hereinafter, also referred to as the “analysis target report”) read by the report reader 30 (S10). The negligence score deriver 44 may extract a plurality of words included in the free description field of the analysis target report using known technology such as morphological analysis. The negligence score deriver 44 counts the number of appearances of each word (“the number of words” in FIG. 5) in the analysis target report.

The negligence score deriver 44 calculates a product (“aggregate value for each word” in FIG. 5) of the number of appearances (“number of words” in FIG. 5) and the negligence term score for each extracted word. The negligence score deriver 44 derives, as the negligence score, a result (quotient) of division with the sum of aggregate values for (“ΣNR” in FIG. 5) for each word of the extracted words as a dividend and the sum of the number of words (“ΣN” in FIG. 5) of the extracted words as a divisor (S12).

When a plurality of analysis target reports are read by the report reader 30, the negligence score deriver 44 repeats the processing of S10 and S12 for each analysis target report and derives the negligence score for each analysis target report (report unit). The negligence score deriver 44 aggregates the negligence scores of the plurality of analysis target reports at various granularities (S14).

For example, the negligence score deriver 44 derives the negligence score for each department (department unit) by obtaining a statistic based on the negligence score of the analysis target report of each department, on the basis of a department name included in each of the plurality of analysis target reports. In other words, the negligence score deriver 44 derives the negligence score of each of the plurality of departments, on the basis of the plurality of incident reports for reporting the incidents having occurred in each of the plurality of departments. In addition, the negligence score deriver 44 derives a negligence score of the entire hospital (hospital unit) by obtaining a statistic based on negligence scores of a plurality of analysis target reports (for example, all analysis target reports) created in a hospital. In other words, the negligence score deriver 44 derives a negligence score of each of a plurality of hospitals, on the basis of a plurality of incident reports for reporting incidents having occurred in each of the plurality of hospitals.

In addition, the negligence score deriver 44 derives a negligence score for each period (for example, for each year/month) by obtaining a statistic based on the negligence score of the analysis target report corresponding to each of a plurality of periods (year unit or month unit), on the basis of an occurrence date included in each of the plurality of analysis target reports. The negligence score deriver 44 derives the negligence score for each year/month for each of the plurality of departments, on the basis of the department name and the occurrence date included in each of the plurality of analysis target reports.

The above statistic may be any one of an average value (an arithmetic mean value or a moving average value), a median value, and a mode value of the negligence scores of the plurality of analysis target reports, or any combination. The statistic may further include a minimum value, a maximum value, a first quartile point, and a third quartile point of the negligence scores of the plurality of analysis target reports. The same is applied to statistics of the severity score, the impact score, and the risk score described later.

The severity score deriver 46 derives a severity score that is an index representing the magnitude of the risk from the viewpoint of a degree of severity of the patient (in other words, seriousness of the incident), on the basis of the severity term score stored in the severity term score storage 24. A method for deriving the severity score is similar to the method for deriving the negligence score illustrated in FIG. 5.

That is, the severity score deriver 46 extracts a plurality of words described in the analysis target report read by the report reader 30, and calculates an aggregate value for each word based on the severity term score, for each of the extracted words (replaces the negligence term score in FIG. 5 with the severity term score). The severity score deriver 46 derives, as the severity score, a result (quotient) of division with the sum of aggregate values (“ΣNR” in FIG. 5) for each of the extracted words as a dividend and the sum of the number of words (“ΣN” in FIG. 5) of the extracted words as a divisor.

When a plurality of analysis target reports are read by the report reader 30, the severity score deriver 46 derives a severity score for each analysis target report (report unit). The severity score deriver 46 aggregates the severity scores of the plurality of analysis target reports at various granularities.

For example, the severity score deriver 46 derives the severity score for each department (department unit) by obtaining a statistic based on the severity score of the analysis target report of each department, on the basis of a department name included in each of the plurality of analysis target reports. In other words, the severity score deriver 46 derives the severity score of each of a plurality of departments, on the basis of the plurality of incident reports for reporting the incident having occurred in each of the plurality of departments. In addition, the severity score deriver 46 derives the severity score of the entire hospital (hospital unit) by obtaining a statistic based on the severity scores of a plurality of analysis target reports (for example, all the analysis target reports) created in a hospital. In other words, the severity score deriver 46 derives the severity score of each of a plurality of hospitals, on the basis of the plurality of incident reports for reporting the incident having occurred in each of the plurality of hospitals.

In addition, the severity score deriver 46 derives the severity score for each period (for example, for each year/month) by obtaining a statistic based on the severity score of the analysis target report corresponding to each of a plurality of periods (year unit or month unit), on the basis of an occurrence date included in each of the plurality of analysis target reports. The severity score deriver 46 derives the severity score for each year/month for each of the plurality of departments, on the basis of the department name and the occurrence date included in each of the plurality of analysis target reports.

The impact score deriver 48 derives an impact score, which is an index representing the magnitude of a risk from the viewpoint of a person determining that the incident report is an incident report in which important contents are described, on the basis of the impact term score stored in the impact term score storage 26. The method for deriving the impact score is similar to the method for deriving the negligence score illustrated in FIG. 5.

That is, the impact score deriver 48 extracts a plurality of words described in the analysis target report read by the report reader 30, and calculates an aggregate value for each word based on the impact term score, for each of the extracted words (replaces the negligence term score in FIG. 5 with the impact term score). The impact score deriver 48 derives, as the impact score, a result (quotient) of division with the sum of aggregate values (“ΣNR” in FIG. 5) for each word of the extracted words as a dividend and the sum of the number of words (“ΣN” in FIG. 5) of the extracted words as a divisor.

When a plurality of analysis target reports are read by the report reader 30, the impact score deriver 48 derives an impact score for each analysis target report (report unit). The impact score deriver 48 aggregates the impact scores of the plurality of analysis target reports at various granularities.

For example, the impact score deriver 48 derives the impact score for each department (department unit) by obtaining a statistic based on the impact score of the analysis target report of each department, on the basis of the department name included in each of the plurality of analysis target reports. In other words, the impact score deriver 48 derives the impact score of each of the plurality of departments, on the basis of the plurality of incident reports for reporting the incident having occurred in each of the plurality of departments. In addition, the impact score deriver 48 derives the impact score of the entire hospital (hospital unit) by obtaining a statistic based on the impact scores of a plurality of analysis target reports (for example, all the analysis target reports) created in a hospital. In other words, the impact score deriver 48 derives the impact score of each of the plurality of hospitals, on the basis of the plurality of incident reports for reporting the incident having occurred in each of the plurality of hospitals.

In addition, the impact score deriver 48 derives the impact score for each period (for example, for each year/month) by obtaining a statistic based on the impact score of the analysis target report corresponding to each of the plurality of periods (year unit or month unit), on the basis of the occurrence date included in each of the plurality of analysis target reports. In addition, the impact score deriver 48 derives an impact score for each year/month for each of the plurality of departments, on the basis of the department name and the occurrence date included in each of the plurality of analysis target reports.

In the present embodiment, a “risk score” is introduced as an index comprehensively representing the magnitude of a risk held by a hospital or each department in the hospital. First, the risk score will be described.

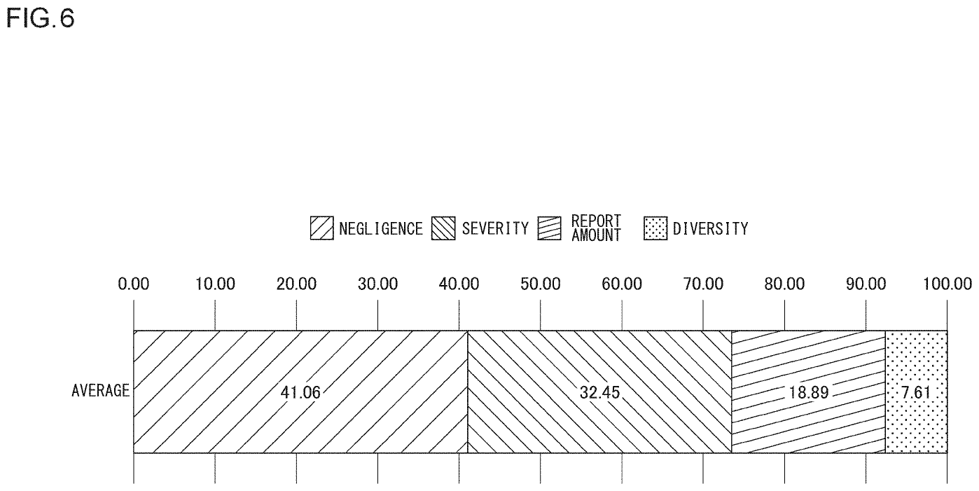

FIG. 6 illustrates weights of risk evaluation factors in a hospital. The same drawing illustrates a result (average value) of weighting by seven evaluators (doctors, nurses, and the like including GRMs) obtained by an analytic hierarchy process (AHP) analysis for a plurality of risk evaluation factors in the hospital. In the result, the weights of negligence and severity among the plurality of risk evaluation factors were large, and a ratio of the weights of negligence and severity was about 4:3. On the basis of this result, the present inventor has selected the negligence score and the severity score as parameter candidates in deriving the risk score.

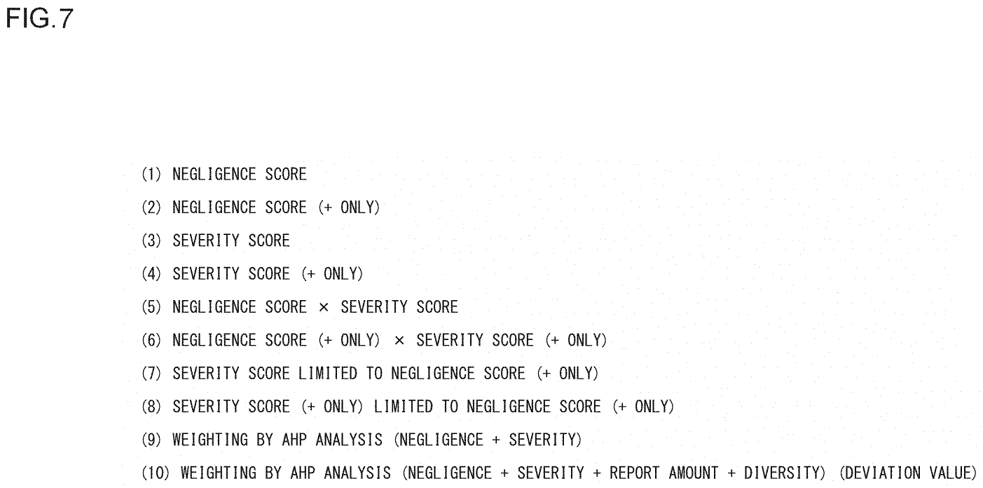

On the other hand, various combinations are conceivable in the negligence score and the severity score. FIG. 7 illustrates candidates for a method for calculating the risk score. In the same drawing, calculation methods (1) to (10) are illustrated. The weighting by the AHP analysis in the calculation methods (9) and (10) is to add the negligence score and the severity score by the ratio (about 4:3) illustrated in FIG. 6.

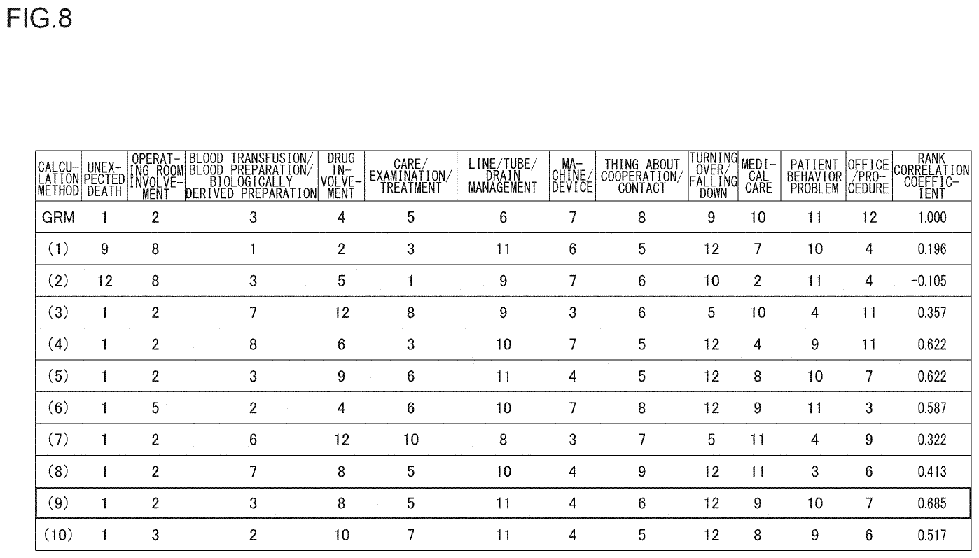

FIG. 8 illustrates a correlation between rank evaluation by GRM and rank evaluation by each calculation method. The present inventor causes seven evaluators (doctors, nurses, and the like including GRMs) to evaluate ranks of a plurality of types of incidents in the hospital. As illustrated in FIG. 8, there are 12 types of incidents including “unexpected death”, “operating room related”, . . . , and “office/procedure”. A GRM line in FIG. 8 indicates ranking results (average values) by the seven evaluators. In addition, lines (1) to (10) in FIG. 8 indicate ranking results for each risk score calculation method illustrated in FIG. 7. As a result of the correlation analysis, it has been found that the calculation method (9) in FIG. 7 has the highest correlation with the determination of the GRM. Therefore, in the embodiment, the risk score is derived using the calculation method (9) in FIG. 7.

Returning to FIG. 4, the risk score deriver 50 derives the risk score on the basis of the negligence term score of each word extracted from the analysis target report (in the embodiment, the negligence score which is a statistic of the negligence scores), the severity term score of each word extracted from the analysis target report (in the embodiment, the severity score which is a statistic of the severity scores), and the weight in the risk evaluation determined in advance for each of the negligence of the hospital and the degree of severity of the patient (in the embodiment, the negligence score:the severity score=4.106:3.245). Specifically, the risk score deriver 50 derives, as the risk score, the sum of a product of the negligence score derived by the negligence score deriver 44 and the weight (4.106) of negligence and a product of the severity score derived by the severity score deriver 46 and the weight (3.245) of severity.

When a plurality of analysis target reports are read by the report reader 30, the risk score deriver 50 derives a risk score for each analysis target report (report unit). The risk score deriver 50 aggregates the risk scores of the plurality of analysis target reports at various granularities.

For example, the risk score deriver 50 derives a risk score for each department (department unit) by obtaining a statistic based on the risk score of the analysis target report of each department, on the basis of the department name included in each of the plurality of analysis target reports. In other words, the risk score deriver 50 derives the risk score of each of a plurality of departments, on the basis of the plurality of incident reports for reporting the incident having occurred in each of the plurality of departments. In addition, the risk score deriver 50 derives a risk score of the entire hospital (hospital unit) by obtaining a statistic based on the risk scores of a plurality of analysis target reports (for example, all the analysis target reports) created in a hospital. In other words, the risk score deriver 50 derives the risk score of each of the plurality of hospitals, on the basis of the plurality of incident reports for reporting the incident having occurred in each of the plurality of hospitals.

In addition, the risk score deriver 50 derives a risk score for each period (for example, for each year/month) by obtaining a statistic based on the risk score of the analysis target report corresponding to each of the plurality of periods (year unit or month unit), on the basis of the occurrence date included in each of the plurality of analysis target reports. For example, the risk score deriver 50 may derive the risk score for each year/month for each of the plurality of departments, on the basis of the department name and the occurrence date included in each of the plurality of analysis target reports.

The outputter 52 outputs a plurality of types of scores derived by the score deriver 42 to a predetermined device or medium. For example, the outputter 52 may store a comma-separated values (CSV) file in which the negligence score, the severity score, the risk score, and the impact score of each hospital have been recorded for each of the plurality of hospitals in a predetermined storage device. In addition, the outputter 52 may store a CSV file in which the negligence score, the severity score, the risk score, and the impact score of each hospital for each year/month (for example, January 2012, February 2012, . . . , etc.) have been recorded in a predetermined storage device.

In addition, the outputter 52 may store a CSV file in which the negligence score, the severity score, the risk score, and the impact score of each department have been recorded for a plurality of departments in the hospital in a predetermined storage device. In addition, the outputter 52 may store a CSV file indicating the negligence score, the severity score, the risk score, and the impact score of each department for each year/month in a predetermined storage device. Note that the outputter 52 may cause a display device to display a plurality of types of scores derived by the score deriver 42.

The image generator 54 generates an image including a statistical chart (statistical graph or the like) based on the plurality of types of scores derived by the score deriver 42. The outputter 52 outputs image data generated by the image generator 54 to a predetermined device or medium. An example of the image generated by the image generator 54 will be described later.

For any of the negligence score, the severity score, the impact score, and the risk score, when a value is larger, it means that a risk held by a hospital or each department in the hospital is higher. According to the risk evaluation device 10 of the first embodiment, it is possible to quantitatively grasp the magnitude of risks held by organizations of various granularities such as hospitals and departments in hospitals, and it is possible to compare the risks between the hospitals or the departments.

The operation of the risk evaluation device 10 according to the first embodiment having the above configuration will be described.

In the classification information storage 20 of the risk evaluation device 10, classification information by the GRM regarding a plurality of incident reports is stored in advance, and the classification information is added and updated every day. When a term score setting instruction designating a plurality of incident reports for setting a term score is input via a user interface or the like, the report reader 30 of the risk evaluation device 10 reads data of the plurality of designated incident reports.

The negligence term score determiner 36 determines the negligence term score for each of the plurality of words described in the plurality of read incident reports and stores the negligence term score in the negligence term score storage 22. The severity term score determiner 38 determines the severity term score for each of the plurality of words described in the plurality of read incident reports and stores the severity term score in the severity term score storage 24. The impact term score determiner 40 determines the impact term score for each of a plurality of words described in the plurality of read incident reports and stores the impact term score in the impact term score storage 26. Note that the negligence term score, the severity term score, and the impact term score may be updated by daily or monthly batch processing.

When a risk score calculation instruction designating a plurality of analysis target incident reports (analysis target reports) and organization information is input via a user interface or the like, the report reader 30 of the risk evaluation device 10 reads the plurality of analysis target reports. In addition, the organization information reader 32 reads the organization information.

The negligence score deriver 44 derives the negligence scores of the plurality of analysis target reports, on the basis of the negligence term scores stored in the negligence term score storage 22. In addition, the negligence score deriver 44 derives the negligence scores of the hospital unit, the department unit, and the year/month unit by aggregating the negligence scores of the plurality of analysis target reports in each of the hospital unit, the department unit, and the year/month unit to obtain a statistic.

The severity score deriver 46 derives the severity scores of the plurality of analysis target reports, on the basis of the severity term score stored in the severity term score storage 24. In addition, the severity score deriver 46 derives the severity scores of the hospital unit, the department unit, and the year/month unit by aggregating the severity scores of the plurality of analysis target reports in each of the hospital unit, the department unit, and the year/month unit to obtain a statistic.

The impact score deriver 48 derives the impact scores of the plurality of analysis target reports, on the basis of the impact term score stored in the impact term score storage 26. In addition, the impact score deriver 48 derives the impact scores of the hospital unit, the department unit, and the year/month unit by aggregating the impact scores of the plurality of analysis target reports in each of the hospital unit, the department unit, and the year/month unit to obtain a statistic.

The risk score deriver 50 derives the risk scores of the plurality of analysis target reports by adding the negligence score and the severity score of each of the plurality of analysis target reports at a predetermined ratio. In addition, the risk score deriver 50 derives the risk scores of the hospital unit, the department unit, and the year/month unit by aggregating the risk scores of the plurality of analysis target reports in each of the hospital unit, the department unit, and the year/month unit to obtain a statistic.

The outputter 52 records the negligence score, the severity score, the impact score, and the risk score of the hospital unit, the department unit, and the year/month unit in a result file, and stores the result file in a predetermined storage area. The image generator 54 generates a predetermined statistical chart on the basis of the negligence score, the severity score, the impact score, and the risk score of the hospital unit, the department unit, and the year/month unit, and generates image data including the statistical chart. The outputter 52 stores the image data generated by the image generator 54 in a predetermined storage area.

Hereinafter, an example of risk evaluation by the risk evaluation device 10 will be described.

The risk score deriver 50 derives the risk score of each of the plurality of hospitals on the basis of the incident report group created in each of the plurality of hospitals. The image generator 54 generates an image in which risk scores of the plurality of hospitals are arranged.

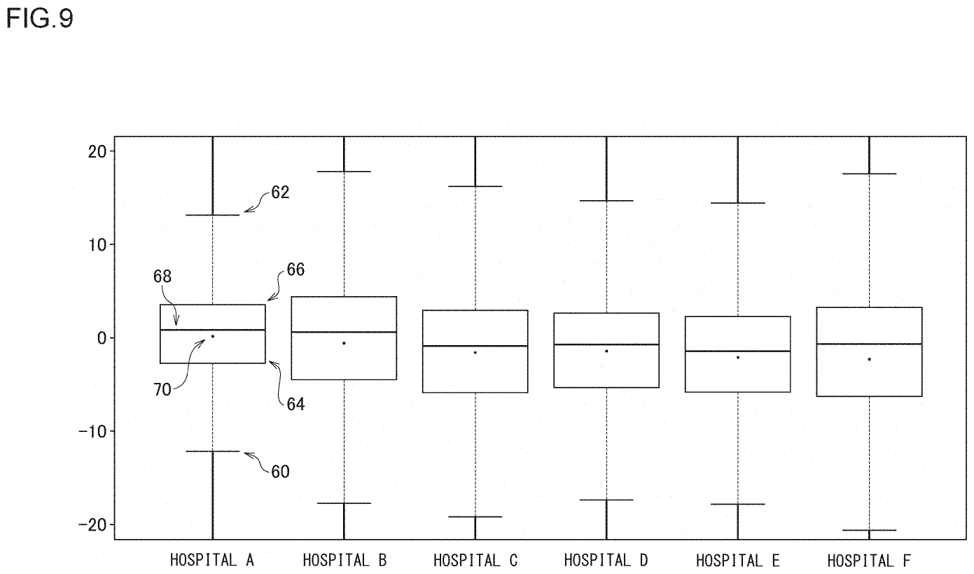

An example of this image is illustrated in FIG. 9. FIG. 9 includes a box plot illustrating risk scores of six hospitals (hospital A to hospital F) as a risk evaluation result. The box plot in FIG. 9 illustrates a minimum value 60, a maximum value 62, a first quartile point 64, a third quartile point 66, a median value 68, and an average value 70 (arithmetic mean value) of the risk scores for a plurality of incident reports for each hospital. As described above, according to the risk evaluation device 10 of the first embodiment, it is easy to visualize the magnitude of risks held by a plurality of different organizations and to compare the risks between the organizations.

In addition, the risk score deriver 50 derives the risk scores of the hospital and each department in each of the plurality of periods, on the basis of a plurality of incident reports for reporting incidents having occurred in each of the plurality of periods, in other words, the incident reports created in each of the plurality of periods. The image generator 54 generates an image indicating variations in risk scores of the hospital and each department over the plurality of periods.

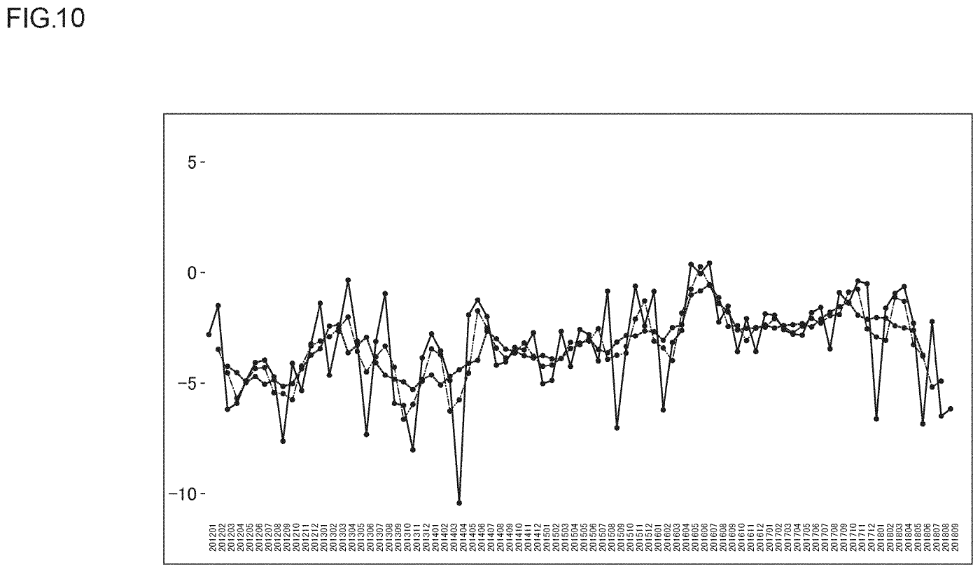

An example of this image is illustrated in FIG. 10. FIG. 10 includes a line graph illustrating a variation in risk score in time series at a department in the hospital as the risk evaluation result. A solid line among elements of the line graph indicates the risk score in the department. A broken line indicates a five-month moving average value of the risk score, and a one-dot chain line indicates a three-month moving average value of the risk score. The image generator 54 may generate an image including a plurality of line graphs illustrating variations in risk scores in time series for a plurality of departments in the hospital. As described above, according to the risk evaluation device 10 of the first embodiment, it is possible to visualize variations in time series for risks held by organizations of various granularities and to perform supporting so that the tendency of the variations can be easily grasped.

### Second Embodiment

Hereinafter, a second embodiment will be described focusing on differences from the first embodiment, and description of common points will be omitted. Of course, features of the second embodiment can be arbitrarily combined with the features of the first embodiment and the modifications.

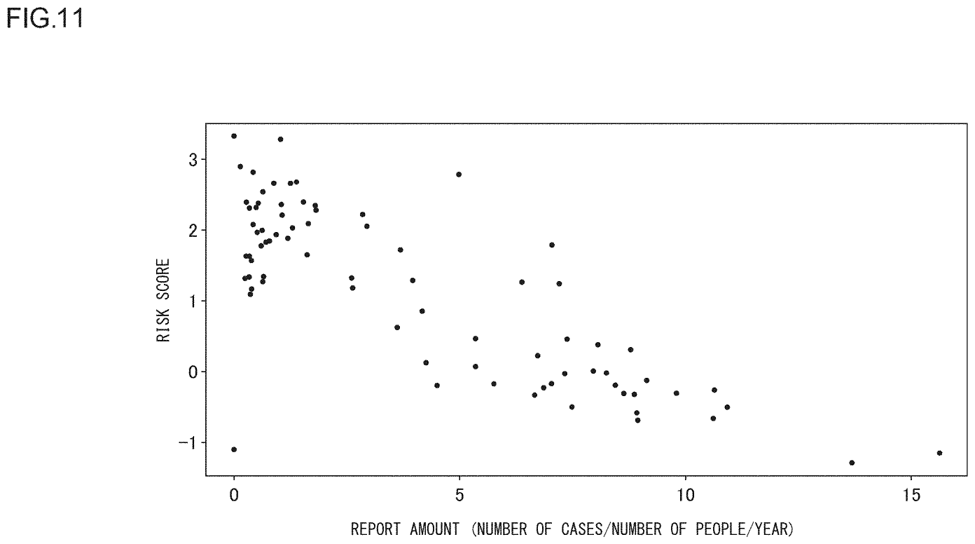

FIG. 11 is a scatter diagram illustrating a relation between a report amount and a risk score. In the scatter diagram, a plurality of points corresponding to a plurality of departments in a hospital are plotted. The report amount on a horizontal axis is the number of incident reports per year and per person in each organization in the hospital. As illustrated in the scatter diagram, there was a tendency that the risk score becomes higher in a department where the report amount is smaller, and the risk score becomes lower in a department where the report amount is larger. This reason is considered to be that there are not many incident reports of high risks even if the report amount is large, but rather, many incident reports of low risks are reported, so that a statistic of scores as a whole becomes low. There were similar tendencies in a negligence score, a severity score, and an impact score.

As described above, the risk score, the negligence score, the severity score, and the impact score are considered to be affected by the report amount (that is, the number of incident reports). Therefore, in the second embodiment, technology for evaluating the magnitude of the risk held by each organization with higher accuracy in consideration of the report amount of each organization is proposed. In the second embodiment, a risk deviation, a negligence deviation, a severity deviation, and an impact deviation are introduced as indices indicating the magnitude of the risk held by each organization in consideration of the report amount of each organization.

Functional blocks of a risk evaluation device 10 of the second embodiment are similar to the functional blocks of the risk evaluation device 10 of the first embodiment illustrated in FIG. 4. A risk score deriver 50 obtains a correspondence relation between the number of incident reports created by each of a plurality of organizations, in other words, the number of incident reports for reporting incidents having occurred in each organization and the risk score of each organization (derived value by the method of the first embodiment). The risk score deriver 50 derives a difference between a standard value of a risk score corresponding to the number of incident reports of an organization and a derived value of the risk score of the organization as a risk deviation of the organization.

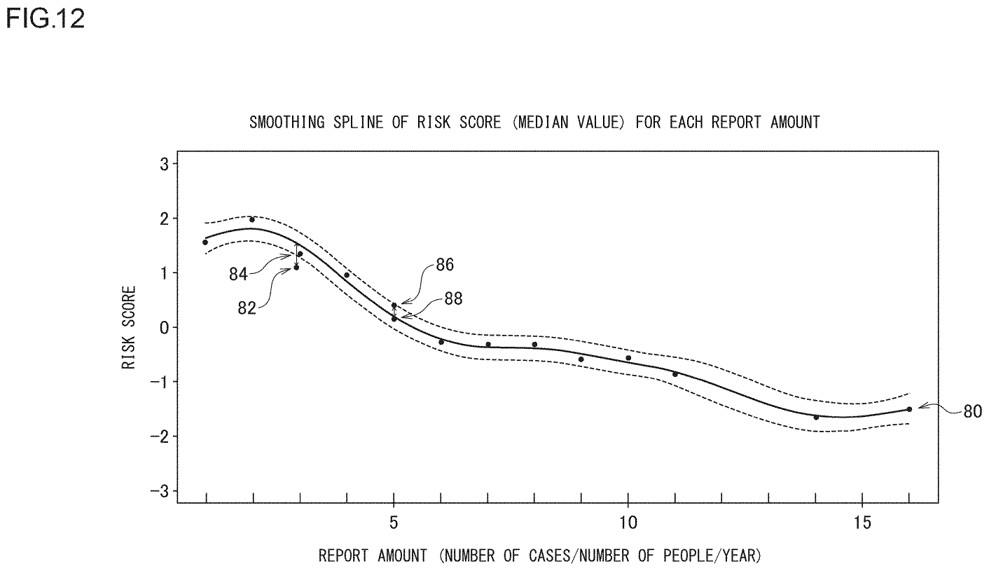

FIG. 12 illustrates a specific example of risk deviation derivation. The risk score deriver 50 acquires the number of people in each organization according to organization information read by an organization information reader 32. The risk score deriver 50 derives the number of incident reports in a unit period in each organization, on the basis of an occurrence date and a department name described in the incident report. The risk score deriver 50 derives a report amount of each organization on the basis of the number of people in each organization and the number of incident reports in the unit period in each organization. The report amount in the second embodiment is the number of incident reports per year and per person.

As a modification, the report amount may be another numerical value based on the number of incident reports. For example, when the number of people of a plurality of organizations to be risk comparison targets is close to each other, the number of incident reports per unit period may be used as the report amount. In addition, when the number of people of the plurality of organizations to be the risk comparison targets and the incident report aggregation period are close to each other, the number of incident reports may be used as the report amount. In addition, an average value, a median value, and a mode value of the number of incident reports of each organization for each unit period may be adopted as the report amount.

The risk score deriver 50 plots positions of the plurality of organizations on a two-dimensional space with the report amount (that is, the number of incident reports per year and per person) as a first axis and the risk score as a second axis, and obtains an approximate curve 80 (also referred to as a spline curve or a smoothing spline) for the position of each organization using a known method such as curve fitting. As the risk score on the second axis, a statistic (median value) of risk scores of a plurality of organizations having the same report amount may be adopted. The approximate curve 80 indicates a standard value of the risk score corresponding to the report amount. Note that broken lines provided above and below the approximate curve 80 in FIG. 12 indicate 95% confidence intervals of the approximate curve.

The risk score deriver 50 identifies the risk score on the approximate curve 80 corresponding to a report amount of an organization as a standard value of the risk score corresponding to the report amount. The risk score deriver 50 derives a difference between the standard value and a derived value (value obtained by the method of the first embodiment, for example, median value) of the risk score of the organization as a risk deviation of the organization. For example, when a department A is plotted on a point 82, a difference 84 between a risk score (standard value) on the approximate curve 80 corresponding to a report amount of the point 82 and a risk score (derived value) of the point 82 is derived as a risk deviation of the department A. In addition, when a department B is plotted on a point 86, a difference 88 between a risk score (standard value) on the approximate curve 80 corresponding to a report amount of the point 86 and a risk score (derived value) of the point 86 is derived as a risk deviation of the department B.

A negligence score deriver 44 derives the negligence deviation by the same method as the risk score deriver 50. That is, the negligence score deriver 44 obtains a correspondence relation between the number of incident reports created by each of a plurality of organizations, in other words, the number of incident reports for reporting incidents having occurred in each organization and the negligence score of each organization. The negligence score deriver 44 derives a difference between a standard value of the negligence score corresponding to the number of incident reports of an organization and a derived value of the negligence score of the organization as the negligence deviation of the organization.

Specifically, the negligence score deriver 44 plots positions of the plurality of organizations on a two-dimensional space with a report amount (number of cases/number of people/year) of the incident report as a first axis and the negligence score as a second axis, and obtains an approximate curve corresponding to the position of each organization. As the negligence score of the second axis, a statistic (median value) of negligence scores of a plurality of organizations of the same report amount may be adopted. It can be said that this approximate curve indicates a standard value of the negligence score corresponding to the report amount. The negligence score deriver 44 identifies the negligence score on the approximate curve corresponding to the report amount of the organization as a standard value of the negligence score corresponding to the report amount. The negligence score deriver 44 derives a difference between the standard value and a derived value (value obtained by the method of the first embodiment, for example, median value) of the negligence score of the organization as the negligence deviation of the organization.

A severity score deriver 46 derives the severity deviation by the same method as the risk score deriver 50. That is, the severity score deriver 46 obtains a correspondence relation between the number of incident reports created by each of the plurality of organizations, in other words, the number of incident reports for reporting incidents having occurred in each organization and the severity score of each organization. The severity score deriver 46 derives a difference between a standard value of the severity score corresponding to the number of incident reports of an organization and a derived value of the severity score of the organization as the severity deviation of the organization.

Specifically, the severity score deriver 46 plots positions of a plurality of organizations on a two-dimensional space with the report amount (number of cases/number of people/year) of the incident report as a first axis and the severity score as a second axis, and obtains an approximate curve corresponding to the position of each organization. As the severity score of the second axis, a statistic (median value) of severity scores of a plurality of organizations of the same report amount may be adopted. It can be said that this approximate curve indicates the standard value of the severity score corresponding to the report amount. The severity score deriver 46 identifies the severity score on the approximate curve corresponding to the report amount of the organization as a standard value of the severity score corresponding to the report amount. The severity score deriver 46 derives a difference between the standard value and a derived value (value obtained by the method of the first embodiment, for example, median value) of the severity score of the organization as the severity deviation of the organization.

An impact score deriver 48 derives the impact deviation by the same method as the risk score deriver 50. That is, the impact score deriver 48 obtains a correspondence relation between the number of incident reports created by each of the plurality of organizations, in other words, the number of incident reports for reporting incidents having occurred in each organization and the impact score of each organization. The impact score deriver 48 derives a difference between a standard value of the impact score corresponding to the number of incident reports of an organization and a derived value of the impact score of the organization as an impact deviation of the organization.

Specifically, the impact score deriver 48 plots positions of the plurality of organizations on a two-dimensional space with the report amount (number of cases/number of people/year) of the incident report as a first axis and the impact score as a second axis, and obtains an approximate curve corresponding to the position of each organization. As the impact score on the second axis, a statistic (median value) of the impact scores of a plurality of organizations of the same report amount may be adopted. It can be said that this approximate curve indicates a standard value of the impact score corresponding to the report amount. The impact score deriver 48 identifies the impact score on the approximate curve corresponding to the report amount of an organization as a standard value of the impact score corresponding to the report amount. The impact score deriver 48 derives a difference between the standard value and a derived value (value obtained by the method of the first embodiment, for example, median value) of the impact score of the organization as the impact deviation of the organization.

According to the risk evaluation device 10 of the second embodiment, the difference between the derived value of the risk index based on the content of the incident report and the standard value of the risk index corresponding to the report amount (the number of incident reports per unit period and number of people) is set as a new risk index value (risk deviation, negligence deviation, severity deviation, and impact deviation). As a result, it is possible to exclude an influence of the number of incident reports of each organization from the risk index value of each organization, and to more accurately evaluate the magnitude of the risk held by each organization.

In addition, according to the risk evaluation device 10 of the second embodiment, the accuracy of risk comparison between a plurality of organizations can be further enhanced. For example, a risk score of a department A (point 82) in FIG. 12 is 1.0, and a risk score of a department B (point 86) is 0.5. Therefore, at first glance, it seems that the risk of the department B is lower than that of the department A. However, a risk deviation of the department A is −0.4 as indicated by a difference 84, and a risk deviation of the department B is 0.3 as indicated by a difference 88. Therefore, it can be determined that the risk of the department A is lower than that of the department B by considering the report amount.

The operation of the risk evaluation device 10 according to the second embodiment will be described.

The negligence score deriver 44 derives a negligence score of each of a plurality of hospitals and a plurality of departments by the same operation as that of the first embodiment. The negligence score deriver 44 further derives a negligence deviation of each of the plurality of hospitals and the plurality of departments, on the basis of the report amount and the negligence score of each of the plurality of hospitals and the plurality of departments. The severity score deriver 46 derives a severity score of each of the plurality of hospitals and the plurality of departments by the same operation as that of the first embodiment. The severity score deriver 46 further derives a severity deviation of each of the plurality of hospitals and the plurality of departments, on the basis of the report amount and the severity score of each of the plurality of hospitals and the plurality of departments.

The impact score deriver 48 derives an impact score of each of the plurality of hospitals and the plurality of departments by the same operation as that of the first embodiment. The impact score deriver 48 further derives an impact deviation of each of the plurality of hospitals and the plurality of departments, on the basis of the report amount and the impact score of each of the plurality of hospitals and the plurality of departments. The risk score deriver 50 derives a risk score of each of the plurality of hospitals and the plurality of departments by the same operation as that of the first embodiment. The risk score deriver 50 further derives a risk deviation of each of the plurality of hospitals and the plurality of departments, on the basis of the report amount and the risk score of each of the plurality of hospitals and the plurality of departments.

In addition to the output data in the first embodiment, an outputter 52 records the negligence deviation, the severity deviation, the impact deviation, and the risk deviation of the hospital unit and the department unit in a result file, and stores the result file in a predetermined storage area. An image generator 54 generates a predetermined statistical chart on the basis of the negligence deviation, the severity deviation, the impact deviation, and the risk deviation of the hospital unit and the department unit, and generates image data including the statistical chart. The outputter 52 stores the image data generated by the image generator 54 in a predetermined storage area.

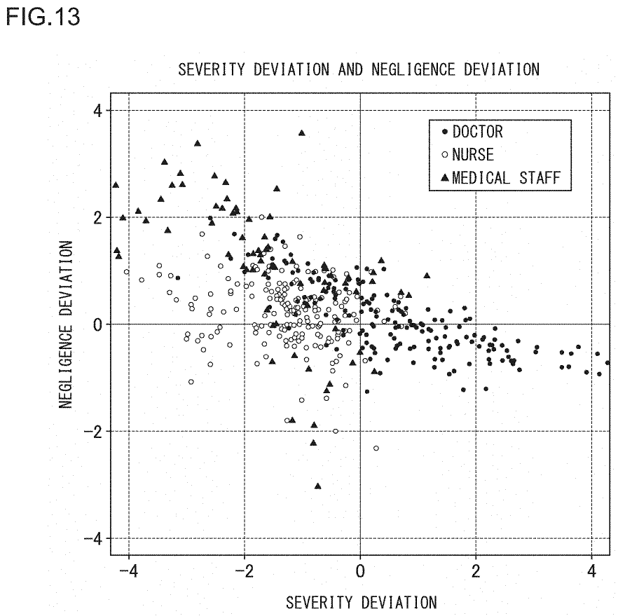

FIG. 13 illustrates an example of an image for risk evaluation generated by the risk evaluation device 10 according to the second embodiment. The image generator 54 generates an image including a scatter diagram in which a position of each of a plurality of organizations (hospitals, departments, and the like) is plotted according to the severity deviation and the negligence deviation of each organization in a two-dimensional space with the severity deviation as a first axis and the negligence deviation as a second axis. In the scatter diagram of the drawing, departments of doctors are indicated by white circles, departments of nurses are indicated by black circles, and departments of medical staffs (pharmacists, radiographers, laboratory technicians, nutritional managers, and the like) are indicated by triangles.

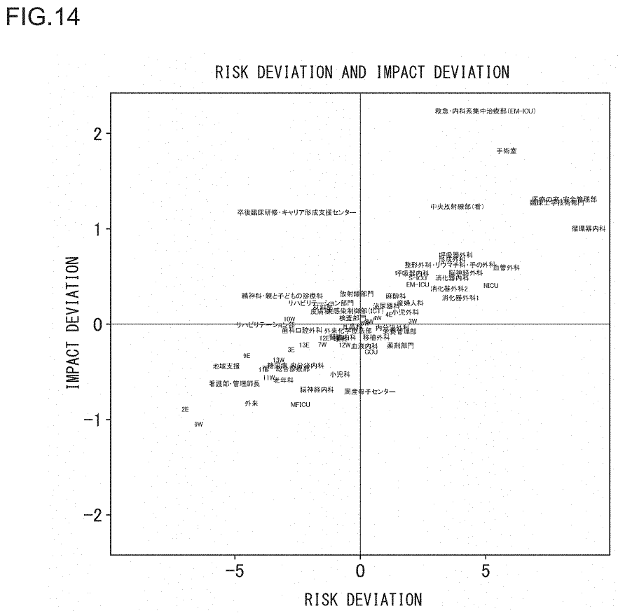

FIG. 14 also illustrates an example of an image for risk evaluation generated by the risk evaluation device 10 according to the second embodiment. The image generator 54 generates an image including a scatter diagram in which a position of each of a plurality of organizations (hospitals, departments, and the like) is plotted according to the risk deviation and the impact deviation of each organization in a two-dimensional space with the risk deviation as a first axis and the impact deviation as a second axis. In the scatter diagram of the drawing, a name of each organization is arranged at the position of each organization.

In the scatter diagram of FIG. 13, it can be determined that organizations arranged in a first quadrant (upper right region) are organizations in which both the severity score and the negligence score are higher than the standard value, and the organizations particularly require attention and support from the viewpoint of safety management. Similarly, in the scatter diagram of FIG. 14, it can be determined that organizations arranged in a first quadrant (upper right region) are organizations in which both the risk score and the impact score are higher than the standard value, and the organizations particularly require attention and support from the viewpoint of safety management.

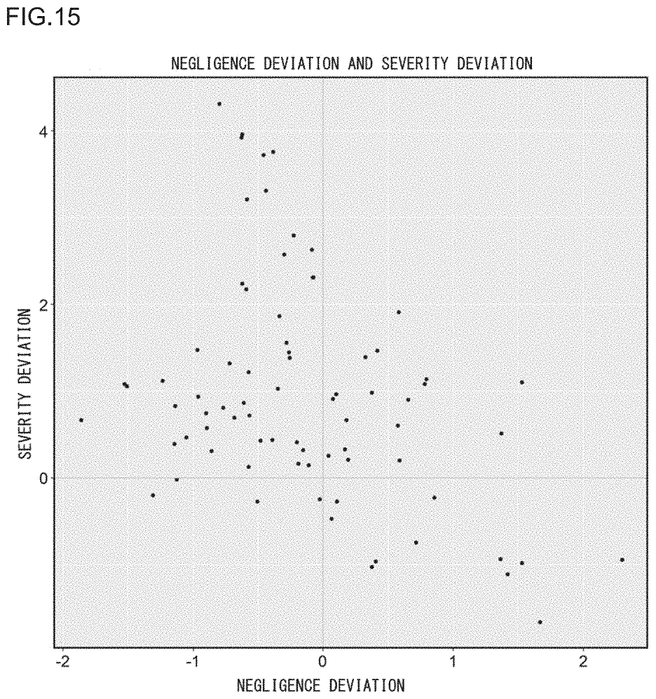

FIG. 15 also illustrates an example of an image for risk evaluation generated by the risk evaluation device 10 according to the second embodiment. FIG. 15 is an image corresponding to FIG. 13, and illustrates an image including a scatter diagram in which a position of each of a plurality of organizations (hospitals, departments, and the like) is plotted according to the severity deviation and the negligence deviation of each organization in the two-dimensional space with the severity deviation as a first axis and the negligence deviation as a second axis. In the scatter diagram in FIG. 13, the position of each organization in a hospital (here, hospital A) is plotted, and in the scatter diagram in FIG. 15, the position of each organization in another hospital (here, hospital B) is plotted. Similarly to the scatter diagram of FIG. 14, in the scatter diagrams of FIGS. 13 and 15, organization names (for example, digestive surgery, pediatrics, outpatient, and the like) may be described.

Comparing the scatter diagram of FIG. 13 regarding the hospital A with the scatter diagram of FIG. 15 regarding the hospital B, the severity deviation and the negligence deviation are within a certain range in both the scatter diagrams, and usefulness of visualizing the risk held by the hospital by the severity deviation and the negligence deviation is confirmed. In addition, according to the risk evaluation device 10 of the second embodiment, it is possible to realize risk evaluation between organizations (for example, between hospitals). For example, by comparing the scatter diagram of FIG. 13 regarding the hospital A with the scatter diagram of FIG. 15 regarding the hospital B, it is possible to easily determine which hospital has a higher risk from the viewpoint of the severity deviation and the negligence deviation, and a factor (department or the like) that increases the risk.

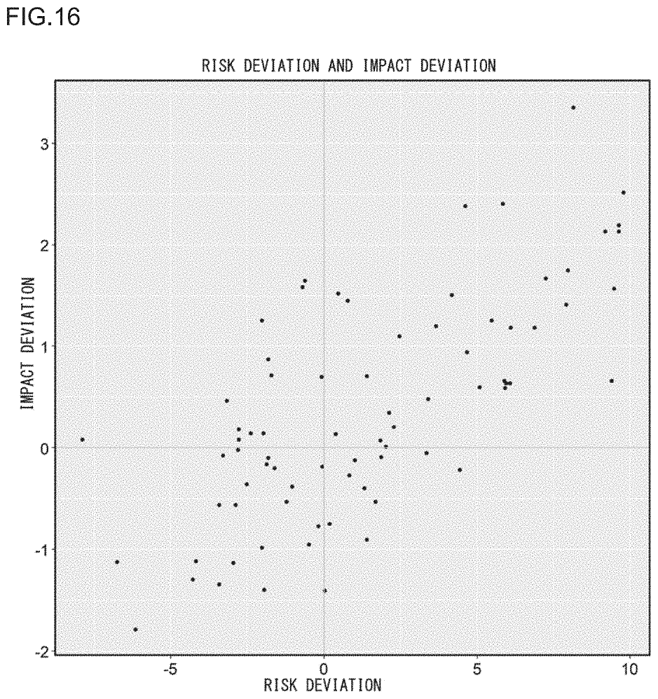

FIG. 16 also illustrates an example of an image for risk evaluation generated by the risk evaluation device 10 according to the second embodiment. FIG. 16 is an image corresponding to FIG. 14, and illustrates an image including a scatter diagram in which a position of each of a plurality of organizations (hospitals, departments, and the like) is plotted according to the risk deviation and the impact deviation of each organization in a two-dimensional space with the risk deviation as a first axis and the impact deviation as a second axis. In the scatter diagram in FIG. 14, the position of each organization in a hospital (here, hospital A) is plotted, and in the scatter diagram in FIG. 16, the position of each organization in another hospital (here, hospital B) is plotted. Similarly to the scatter diagram of FIG. 14, in the scatter diagram of FIG. 16, organization names may be described.

Comparing the scatter diagram of FIG. 14 regarding the hospital A with the scatter diagram of FIG. 16 regarding the hospital B, the risk deviation and the impact deviation are within a certain range in both the scatter diagrams, and usefulness of visualizing the risk held by the hospital by the risk deviation and the impact deviation is confirmed. In addition, according to the risk evaluation device 10 of the second embodiment, it is possible to realize risk evaluation between organizations (for example, between hospitals). For example, by comparing the scatter diagram of FIG. 14 regarding the hospital A with the scatter diagram of FIG. 16 regarding the hospital B, it is possible to easily determine which hospital has a higher risk from the viewpoint of the risk deviation and the impact deviation and a factor (department or the like) that increases the risk.

Note that the image generator 54 of the risk evaluation device 10 may generate an image obtained by adding an object (referred to as a “reference object”) indicating a reference value or range to the scatter diagrams of FIGS. 13 to 16. The reference value or range may be a minimum value, a maximum value, or a range from the minimum value to the maximum value of the reference hospital or the comparison hospital. For example, the reference object may be a rectangular object indicating a range from the minimum value to the maximum value of the risk deviation (severity deviation) of the reference hospital and a range from the minimum value to the maximum value of the impact deviation (negligence deviation) of the reference hospital. In addition, the reference value or range may be an average value of minimum values, an average value of maximum values, or a range from the average value of the minimum values to the average value of the maximum values across a plurality of hospitals. According to this configuration, it is possible to more easily evaluate the risk held by each organization and compare the risks between the organizations.

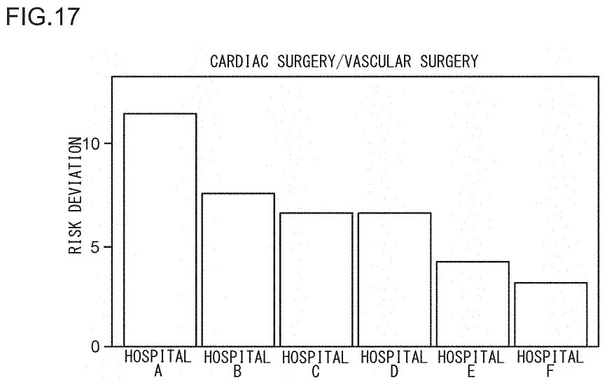

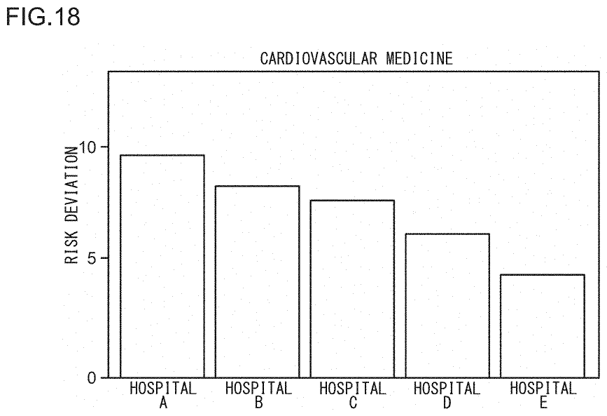

FIGS. 17 and 18 also illustrate examples of images for risk evaluation generated by the risk evaluation device 10 according to the second embodiment. The image generator 54 of the risk evaluation device 10 derives risk deviations of a plurality of hospitals to be compared, on the basis of the incident reports of the plurality of hospitals, and generates an image including a graph (for example, a bar graph) indicating the risk deviations of the plurality of hospitals side by side. FIG. 17 illustrates risk deviations for cardiac surgery and vascular surgery of each hospital derived on the basis of incident reports of cardiac surgery and vascular surgery of the plurality of hospitals. In addition, FIG. 18 illustrates risk deviations for cardiovascular medicine of each hospital derived on the basis of incident reports of cardiovascular medicine of the plurality of hospitals. According to this configuration, it is possible to further facilitate risk comparison between organizations (here, between hospitals).

The present disclosure has been described above on the basis of the first embodiment and the second embodiment. These embodiments are merely examples, and it is understood by those skilled in the art that various modifications can be made in the combination of the respective components or the respective processes, and that the modifications are also within the scope of the present disclosure.

Although not mentioned in the first embodiment and the second embodiment, as a modification, the risk evaluation device 10 may further derive a core score and perform risk evaluation based on the core score.

Specifically, the classification information storage 20 of the risk evaluation device 10 may store classification information indicating whether or not each of a plurality of incident reports created in the hospital corresponds to an incident report determined by a meeting of people (hereinafter, also referred to as a “board”) that important contents are described from the viewpoint of grasping and evaluating the risk. A set of corresponding incident reports is referred to as a “core report group”, and a set of non-corresponding incident reports is referred to as a “non-core report group”. The board checks the contents of the plurality of incident reports and determines whether each incident report corresponds to the core report group or the non-core report group. The classification information indicating this determination result may be stored in the classification information storage 20. Note that the board may have members different from the GRM that determines the impact report, and the determination of the core report may be different from the determination of the impact report.

The term score determiner 34 of the risk evaluation device 10 may further include a core term score determiner. The core term score is similar to the impact term score. The core term score is an index representing the likelihood of appearance in an incident report determined by the board that important contents are described from the viewpoint of grasping and evaluating the risk, in other words, an incident report determined by the board that careful consideration or some action is necessary. A method for deriving the core term score may be similar to the method for deriving the impact term score illustrated in FIG. 3, and may be a method in which the “impact report group” in FIG. 3 is replaced with the “core report group” and the “non-impact report group” in FIG. 3 is replaced with the “non-core report group”. The core term score of each word derived by the core term score determiner may be stored in the storage 14 (core term score storage).

The score deriver 42 of the risk evaluation device 10 may further include a core score deriver. The core score deriver derives the core score on the basis of the core term score stored in the core term score storage. The core score is similar to the impact score, and is an index representing the magnitude of the risk from the viewpoint of the board determining that the report is an incident report in which important contents are described. A method for deriving the core score is similar to the method for deriving the negligence score illustrated in FIG. 5 (the negligence term score in FIG. 5 is replaced with the core term score). When a plurality of analysis target reports are read by the report reader 30, the core score deriver derives the core score for each analysis target report (report unit). The core score deriver aggregates the impact scores of the plurality of analysis target reports at various granularities (hospital unit, department unit, or the like). Similarly to the impact score deriver, the core score deriver derives core scores in organizations of various granularities or in various periods.

In addition, the core score deriver derives a core deviation by a method similar to that of the risk score deriver 50. That is, the core score deriver obtains a correspondence relation between the number of incident reports created by each of a plurality of organizations, in other words, the number of incident reports for reporting incidents having occurred in each organization and the core score of each organization. The core score deriver derives a difference between a standard value of the core score corresponding to the number of incident reports of an organization and a derived value of the core score of the organization as the core deviation of the organization.

The outputter 52 may further record core scores and core deviations of the hospital unit, the department unit, and the year/month unit in the result file. For example, the outputter 52 may generate and record a document file indicating the negligence score, the severity score, the impact score, the risk score, and the core score of each of the plurality of incident reports. The image generator 54 may generate a predetermined statistical chart including core scores or core deviations of the hospital unit, the department unit, and the year/month unit, and generate image data including the statistical chart. For example, the image generator 54 may generate an image including a scatter diagram in which the impact deviations in FIGS. 14 and 16 are replaced with a core deviation.

By using the core score and the core deviation, the risk of the organization can be evaluated from more various viewpoints. Similarly to the impact score, the core score is based on human determination, but is based on a result of consultation by a plurality of people unlike the impact score. Therefore, by introducing the core score and the core deviation, it is possible to suppress occurrence of leakage in the risk evaluation and to support more appropriate risk evaluation and appropriate determination as an organization.

Note that the risk evaluation device 10 derives the severity score (severity deviation), the negligence score (negligence deviation), the risk score (risk deviation), and the core score (core deviation), but may not derive the impact score (impact deviation). This is because there may be an organization in which determination by the GRM is not obtained.

The risk evaluation devices 10 described in the first embodiment and the second embodiment may include a communication device that communicates with an external device in accordance with a predetermined communication protocol, and may provide a risk evaluation service as a cloud service or software as a service (SaaS). For example, a client device may upload the incident report and the organization information to the risk evaluation device 10 and request the risk evaluation device 10 to evaluate the risk. The controller 12 of the risk evaluation device 10 may acquire the incident report and the organization information via the communication device and derive scores of various risk indices. The outputter 52 of the risk evaluation device 10 may transmit a risk evaluation result (risk score, risk deviation, statistical chart, and the like) to the client device via the communication device.

The plurality of functional blocks of the risk evaluation device 10 described in the first embodiment and the second embodiment may be distributed and provided in a plurality of devices. In this case, the plurality of devices may communicate with each other and cooperate as a system, thereby exerting the same function as the risk evaluation device 10 in the first embodiment and the second embodiment.

The technical ideas (risk evaluation technologies) described in the first embodiment and the second embodiment can be applied to cases other than the case of analyzing the incident report created in the medical institution and evaluating the risk held by the medical institution. For example, the report to be analyzed may be an incident report in various industries such as the transportation industry and the construction industry. Further, the report is not limited to the incident report, and may be a near miss report, a paper, or a news article. The technical ideas described in the first embodiment and the second embodiment can be widely applied to the case of estimating and visualizing the risk held by the organization in which the event indicated by the report has occurred.

Any combination of the above-described embodiments and modifications is also useful as an embodiment of the present invention. The new embodiment generated by the combination has the effect of each of the combined embodiments and modifications. In addition, it is understood by those skilled in the art that the functions to be performed by the components described in the claims are realized by single bodies of the components described in the embodiments and the modifications or by cooperation of the components.

### INDUSTRIAL APPLICABILITY

The technology of the present disclosure can be applied to a system or device related to risk evaluation.

### REFERENCE SIGNS LIST

10 risk evaluation device, 22 negligence term score storage, 24 severity term score storage, 26 impact term score storage, 30 report reader, 34 term score determiner, 42 score deriver, 52 outputter, 54 image generator

1. A risk evaluation system for analyzing a report for reporting an event having occurred in an organization and evaluating the magnitude of a risk of the organization, the risk evaluation system comprising:
a first storage structured to store, for each of a plurality of words, a negligence term score that is an index representing the likelihood of appearance in a report indicating that an event has occurred due to negligence of the organization;
a second storage structured to store, for each of a plurality of words, a seriousness term score that is an index representing the likelihood of appearance in a report indicating that a serious event has occurred; and
a score deriver structured to derive a risk score on the basis of the negligence term score of each word described in the report to be analyzed, the seriousness term score of each word described in the report to be analyzed, and a weight in risk evaluation determined in advance for each of the negligence of the organization and the seriousness of the event.

2. The risk evaluation system according to claim 1, wherein the score deriver derives a plurality of risk scores on the basis of a plurality of reports for reporting an event having occurred in the organization, and derives a statistic based on the plurality of risk scores as the risk score of the organization.

3. The risk evaluation system according to claim 1, further comprising:
an image generator, wherein
the score deriver derives a risk score of each of a plurality of organizations on the basis of the plurality of reports for reporting an event having occurred in each of the plurality of organizations, and
the image generator generates an image indicating the risk score of each of the plurality of organizations.

4. The risk evaluation system according to claim 1, further comprising:
an image generator, wherein
the score deriver derives a risk score of the organization in each of a plurality of periods, on the basis of a plurality of reports for reporting an event having occurred in each of the plurality of periods, and
the image generator generates an image indicating a variation in the risk score of the organization over the plurality of periods.

5. The risk evaluation system according to claim 1, wherein
the score deriver derives a risk score of each of a plurality of organizations on the basis of the plurality of reports for reporting an event having occurred in each of the plurality of organizations, and
the score deriver obtains a correspondence relation between the number of reports and a standard risk score on the basis of the number of reports and the risk score of each of the plurality of organizations, and further derives a difference between a standard value of a risk score corresponding to the number of reports of an organization and a derived value of the risk score of the organization as a risk deviation of the organization.

6. The risk evaluation system according to claim 5, further comprising:
a third storage structured to store, for each of a plurality of words, an impact term score that is an index representing the likelihood of appearance in a report determined by a person that important contents are described; and
an image generator, wherein
the score deriver further derives an impact score that is an index representing the magnitude of a risk of each of the plurality of organizations, on the basis of an impact term score of each word described in the plurality of reports for reporting an event having occurred in each of the plurality of organizations,
the score deriver obtains a correspondence relation between the number of reports and a standard impact score on the basis of the number of reports and the impact score of each of the plurality of organizations, and further derives a difference between a standard value of an impact score corresponding to the number of reports of an organization and a derived value of the impact score of the organization as an impact deviation of the organization, and
the image generator generates an image in which a position of each organization is plotted according to the risk deviation and the impact deviation of each organization in a two-dimensional space with the risk deviation as a first axis and the impact deviation as a second axis.

7. A risk evaluation method in which a computer accessible to a first storage and a second storage analyzes a report for reporting an event having occurred in an organization and evaluates the magnitude of a risk of the organization, the method comprising:
causing the first storage to store, for each of a plurality of words, a negligence term score that is an index representing the likelihood of appearance in a report indicating that an event has occurred due to negligence of the organization,
causing the second storage to store, for each of a plurality of words, a seriousness term score that is an index representing the likelihood of appearance in a report indicating that a serious event has occurred,
extracting the plurality of words described in the report to be analyzed; and
deriving a risk score that is an index representing the magnitude of the risk of the organization, on the basis of the negligence term score of each of the extracted words, the seriousness term score of each of the extracted words, and a weight in risk evaluation determined in advance for each of the negligence of the organization and the seriousness of the event.

8. A non-transitory computer-readable storage medium storing a computer program for causing a computer accessible to a first storage and a second storage to analyze a report for reporting an event having occurred in an organization and evaluate the magnitude of a risk of the organization, wherein
the first storage stores, for each of a plurality of words, a negligence term score that is an index representing the likelihood of appearance in a report indicating that an event has occurred due to negligence of the organization,
the second storage stores, for each of a plurality of words, a seriousness term score that is an index representing the likelihood of appearance in a report indicating that a serious event has occurred, and wherein
the computer program causing the computer to realize:
a function of extracting the plurality of words described in the report to be analyzed; and
a function of deriving a risk score that is an index representing the magnitude of the risk of the organization, on the basis of the negligence term score of each of the extracted words, the seriousness term score of each of the extracted words, and a weight in risk evaluation determined in advance for each of the negligence of the organization and the seriousness of the event.
